(19) United States
(12) Patent Application Publication
(10) Pub. No.: US 2026/0240421 A1
(43) Pub. Date: Aug. 20, 2026

(54) URETEROSCOPE CONFIGURED TO CHANGE A RIGIDITY OF A DISTAL END REGION OF A CATHETER AND METHODS OF USING THE SAME

(71) Applicant: C.R. BARD, INC., Murray Hill, NJ (US)

(72) Inventors: Tracey Knapp, Snellville, GA (US); Justin Wolfe, Lawrenceville, GA (US); Cecille Canary, Atlanta, GA (US)

(21) Appl. No.: 18/843,914

(22) PCT Filed: Mar. 9, 2022
(86) PCT No.: PCT/US2022/019579

§ 371 Date: Sep. 4, 2024

(51) Int. Cl. A61B 1/005; A61B 1/00; A61B 1/018

(52) U.S. Cl. CPC A61B 1/0052; A61B 1/00042; A61B 1/00128; A61B 1/0055; A61B 1/0057; A61B 1/018; A61B 1/00045

(57) ABSTRACT

An example ureteroscope includes a handpiece having a catheter end and a control end. The ureteroscope also includes a catheter extending from the catheter end of the handpiece. The ureteroscope is configured to selectively change the rigidity of a distal end region of the catheter. For instance, the ureteroscope is configured to increase the rigidity of the distal end of the catheter.

### BACKGROUND

Endoscopes of small size are desired in many industrial and medical applications. For example, when natural orifices and lumens of a human body are small, small endoscopes are used for insertion through such orifices and lumens to target locations within the body. For single incision laparoscopy, smaller endoscopes are preferred to provide an inside-the-body view of the surgical site, particularly when the incision itself is of minimal dimensions. Sometimes, patients may feel irritation when an endoscope is being inserted into his or her body, and a smaller endoscope may mitigate such unpleasant experience and may minimize trauma to the patient. Moreover, a physician may improve diagnostic and procedural protocols with a smaller endoscope. For example, transnasal endoscopy may sometimes replace trans-oral endoscopy.

### SUMMARY

Embodiments are directed to ureteroscopes, endoscopic systems including the same, and methods of using the same. In an embodiment, a ureteroscope is disclosed. The ureteroscope includes a handpiece. The handpiece includes a catheter end, a control end, working assembly configured to receive one or more surgical instruments, and steering controls configured to bend at least a portion of the catheter. The ureteroscope also includes a catheter extending from the catheter end of the handpiece. The catheter includes a proximal end region and a distal end region spaced further from the handpiece than the proximal end region. At least one of the working assembly includes a first moveable portion configured to move relative to the catheter end of the handpiece or the steering controls includes a second moveable portion configured to move relative to the catheter end of the handpiece. Movement of the first moveable portion relative to the catheter end of the handpiece changes a rigidity of at least a portion of the catheter. Movement of the second moveable portion relative to the catheter end of the handpiece changes a rigidity of at least a portion of the catheter.

In an embodiment, a method of using a ureteroscope is disclosed. The method includes providing a ureteroscope. The ureteroscope includes a handpiece. The handpiece includes a catheter end, a control end, a working assembly configured to receive one or more surgical instruments, and steering controls configured to bend at least a portion of the catheter. The ureteroscope also includes a catheter extending from the catheter end of the handpiece. The catheter includes a proximal end region and a distal end region spaced further from the handpiece than the proximal end region. At least one of the working assembly includes a first moveable portion configured to move relative to the catheter end of the handpiece or the steering controls includes a second moveable portion configured to move relative to the catheter end of the handpiece. The method also includes moving at least one of at least a portion of the first moveable portion or at least a portion of the second moveable portion relative to the catheter end of the handpiece of the ureteroscope to adjust a rigidity of at least a portion of the catheter.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1A is an isometric and schematic view of an endoscopic system, according to an embodiment.

FIG. 1B is a cross-sectional view of a portion of the ureteroscope taken along plane 1B-1B.

FIG. 1C is a side elevational view of the terminal end of the distal end region of the catheter, according to an embodiment.

FIGS. 1D and 1E are enlarged views of the ureteroscope illustrating a method of selectively changing a rigidity of at least a portion of the catheter using the actuator, according to an embodiment.

FIGS. 2A and 2B are side elevational views of a ureteroscope configured to use the steering controls to change the rigidity at least a portion of the catheter, according to an embodiment.

FIG. 3 is a side elevational view of a catheter shaft that may be used in any of the catheters disclosed herein, according to an embodiment.

### DETAILED DESCRIPTION

Embodiments are directed to ureteroscopes, endoscopic systems including the same, and methods of using the same. An example ureteroscope includes a handpiece having a catheter end and a control end. The ureteroscope also includes a catheter extending from the catheter end of the handpiece. The handpiece also includes a working assembly configured to engage with one or more surgical instruments and steering controls configured to selectively bend at least a portion of the catheter. The ureteroscope is configured to selectively change the rigidity of at least a portion of the catheter. In an example, at least one of the working channel port includes a first moveable portion or the steering controls includes a second moveable portion. The first moveable portion and/or the second moveable portion are configured to move relative to a portion of the handpiece (e.g., the catheter end). Moving the first moveable portion and/or the second moveable portion relative to a portion of the handpiece may selectively change the rigidity of at least a portion of the catheter.

Some ureteroscopes include catheters having flexible distal end regions (i.e., regions of the catheter distal to the handpiece thereof). The flexible distal end regions of such catheters allow the catheters to move through convoluted pathways in a human or animal body. The flexible distal end regions of such catheters also allows the shape (e. g., curvature) of the catheters to be selectively changed thereby steering the catheter through bends in the convoluted pathways or to choose which passageways to move the catheter through. However, the flexible distal ends of the catheters of such ureteroscopes may buckle or be unable to move through tight passageways or at least partially obstructed passageways due to the flexibility thereof.

Unlike the ureteroscopes discussed in the preceding paragraph, the ureteroscopes disclosed herein are able to selectively change the rigidity of at least a portion of the catheters thereof. For example, the ureteroscopes disclosed herein may include a catheter having a flexible distal end region during normal use. However, the rigidity of the distal end region of the catheter may be selectively increased (i.e., the flexibility of the distal end region may be selectively decreased) when the pathway through the body narrows, the pathway is at least partially obstructed, or for any other reason. Increasing the rigidity of the distal end region of the catheter allows the distal end region of the catheter to push through the narrowed pathway or push the obstacle aside. After moving the distal end region of the catheter through the narrowed pathway or after pushing aside the obstacle, the rigidity of the distal end region of the catheter may be selectively decreased (i.e., the flexibility of the distal end region may be selectively increased).

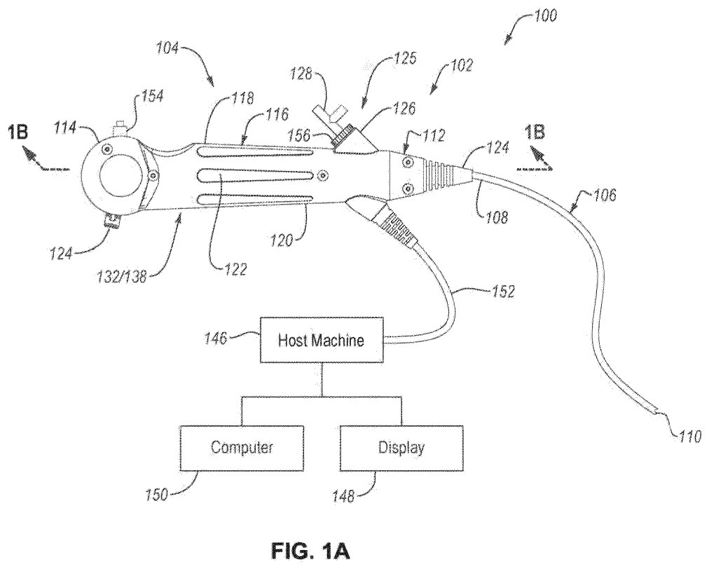

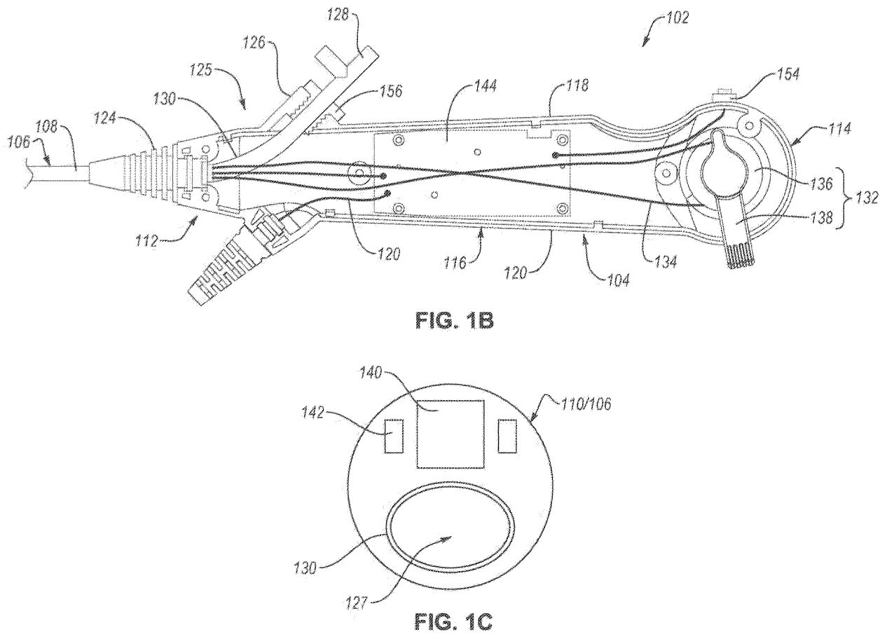

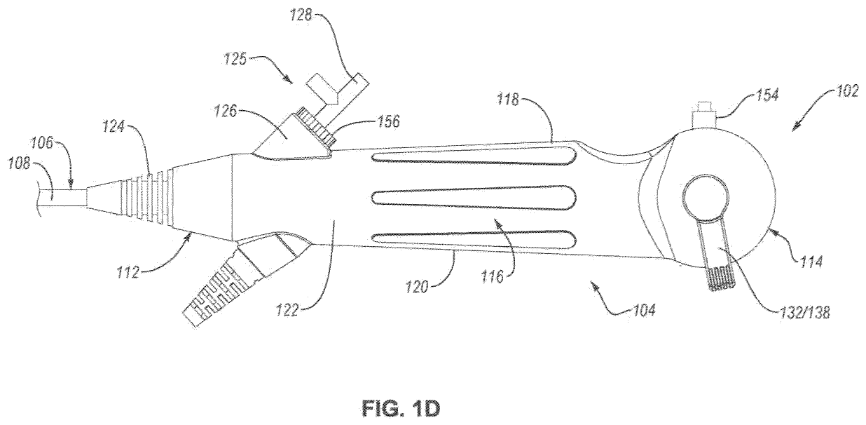

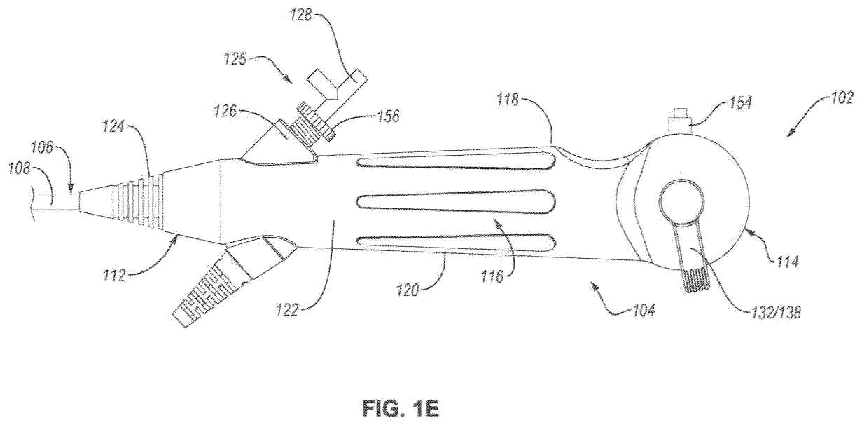

FIG. 1A is an isometric and schematic view of an endoscopic system 100, according to an embodiment. The endoscopic system 100 includes a ureteroscope 102. FIG. 1B is a cross-sectional view of a portion of the ureteroscope 102 taken along plane 1B-1B. The ureteroscope 102 includes a handpiece 104 and a catheter 106 extending from the handpiece 104. The catheter 106 includes a proximal end region 108 adjacent to or within the handpiece 104 and a distal end region 110 opposite the proximal end region 108. The ureteroscope 102 is configured to selectively change the rigidity of at least a portion of the catheter 106 (e. g., the distal end region 110 of the catheter 106).

The handpiece 104 may be elongated and includes a catheter end 112 and a control end 114. The catheter end 112 is proximate to the catheter 106 and a control end 114 opposite or distal to the catheter end 112. As the control end 114 is opposite to the catheter end 112, the control end 114 and the catheter end 112 are at different ends of the handpiece 104 and may face different directions relative to one another. The control end 114 may include a generally bulbous shape, and the handpiece 104 may taper between the control end 114 and the catheter end 112.

With the control end 114 opposite or distal to the catheter end 112, the handpiece 104 includes an intermediate or central portion 116 positioned between the catheter end 112 and the control end 114. The central portion 116 includes a first or top surface 118, a second or bottom surface 120, and two opposing sides 122 positioned between the top surface 118 and the bottom surface 120. In an embodiment, the top and bottom surfaces 118, 120 of the central portion 116 are rounded and the sides 122 are substantially flat.

In an embodiment, both the catheter 106 and the handpiece 104 are disposable. In some embodiments, the catheter 106 and the handpiece 104 are manufactured as an integral part, or the catheter 106 is fixed with the handpiece 104 via a handpiece-catheter connector 124. Alternatively, only the catheter 106 is disposable, and the handpiece 104 may be sterilized and reused multiple times. In this case, the catheter 106 is removably connected to the handpiece 104 and the catheter end 112 via a handpiece-catheter connector 124.

As previously discussed, the catheter 106 includes a proximal end region 108 and a distal end region 110 spaced from the proximal end region 108. The proximal end region 108 is attached to, integrally formed with, or disposed in the catheter end 112 (e.g., the handpiece-catheter connector 124). The distal end region 110 may be configured to be disposed in the urethral opening of an individual. FIG. 1C is a side elevational view of the terminal end of the distal end region 110 of the catheter 106, according to an embodiment.

The ureteroscope 102 includes a working assembly 125. The working assembly 125 includes a working channel port 126 defined by the handpiece 104 and a working channel 127 defined by or disposed within the catheter 106 (FIG. 1C). The working channel 127 extends from the proximal end region 108 to the distal end region 110 of the catheter 106. The working channel port 126 and the working channel 127 are connected together (either directly or indirectly). The working channel port 126 and the working channel 127 are configured to engage with various surgical instruments and irrigation devices, as needed, for operations such as stone breaking and retrieval, etc. For example, the various surgical instruments may be inserted through the working channel port 126 and through the working channel 127 such that the various surgical instruments may be used at the distal end region 110 of the catheter 106. The working assembly 125 may also include at least one of a working channel connector 128 that is connected or attached to the working channel port 126 or at least one conduit 130 that indirectly connects the working channel port 126 to the catheter 106. In an embodiment, as shown, the conduit 130 may extend within the catheter 106 and define at least a portion of the working channel 127. In such an embodiment, the conduit 130 may extend through the catheter 106 and be rigidly (e.g., non-moveably) attached to at least the distal end region 110 of the catheter 106. In an embodiment, the conduit 130 does not extend within the catheter 106 and, instead, the catheter 106 defines the working channel 127.

The working channel port 126 may be positioned proximate to the catheter end 112 of the handpiece 104. For example, the working channel port 126 may be positioned less than one-half of a distance from the catheter end 112 to the control end 114, less than one-third of the distance from the catheter end 112 to the control end 114, less than one-quarter of the distance from the catheter end 112 to the control end 114, or less than one-fifth of the distance from the catheter end 112 to control end 114. Positioning the working channel port 126 proximate to the catheter end 112 may improve the ergonomics of the handpiece 104 since the working channel port 126 and any surgical instruments extending therefrom are less likely to interfere with use of the ureteroscope 102.

The ureteroscope 102 includes steering controls 132 that are configured to control one or more steering wires 134 that are connected to an active bend portion of the catheter 106 (e.g., distal end region 110) to deflect the active bend portion of the catheter 106 to a desired location. Accordingly, a user may bend or curve the catheter 106 (e.g., bend or curve the distal end region 110) using the steering controls 132.

The steering controls 132 may include a driver 136 that is coupled to the steering wires 134. The driver 136 is configured to move the steering wires 134 thereby bending the catheter 106. For example, the driver 136 is configured to rotate about a rotation axis. The rotation axis may extend generally perpendicular to a longitudinal axis of handpiece 104. The steering wires 134 may be attached to opposing sides of the driver 136. As such, rotation of the driver 136 about the rotation axis may cause at least one of the steering wires 134 to move closer to or further away from the catheter end 112 of the handpiece 104. In an example, when the steering wires 134 include two steering wires 134, as shown, rotation of the driver 136 may cause one of the steering wires 134 to move closer to the catheter end 112 while the other steering wire 134 moves further from the catheter end 112. In such an example, the steering wire 134 that moves closer to the catheter end 112 is not in tension while the steering wire 134 that moves further to the catheter end 112 is in tension. Selectively causing one of the two steering wires 134 to be in tension causes at least a portion of the catheter 106 to selectively bend. It is noted that the principles of selectively tensioning one or more of the steering wires 134 is what causes the catheter 106 to selectively bend or curve, regardless if the ureteroscope 102 includes one or three of more steering wires 134.

In an example, the driver 136 may exhibit a generally cylindrical shape which allows the driver 136 to fit within the generally bulbous control end 114. However, it is noted that the driver 136 may exhibit other shapes.

In an example, as illustrated, the driver 136 is disposed in an interior of the handpiece 104. In an example, the driver 136 is at least partially disposed on or otherwise accessible from an exterior of the handpiece 104.

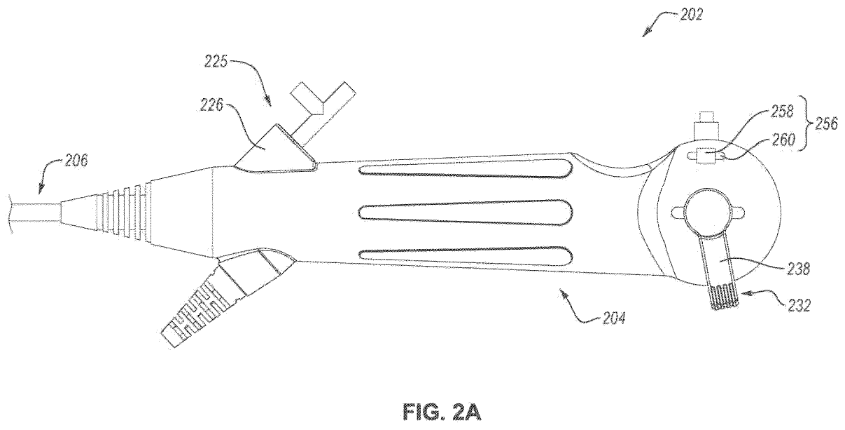

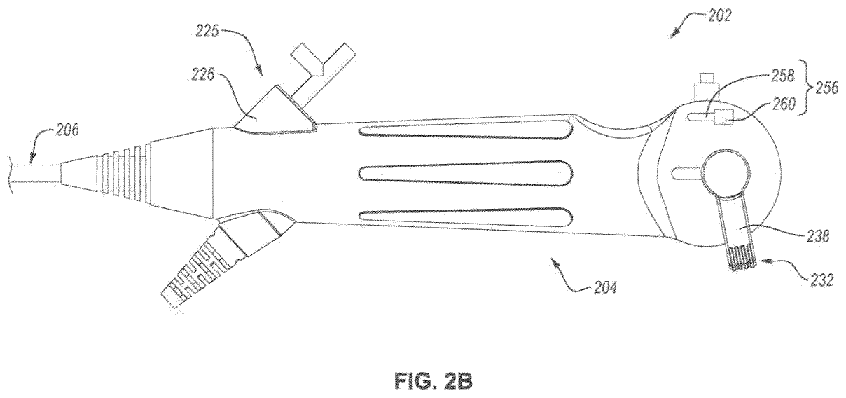

It is noted that the steering controls 132 may include one or more elements instead of the driver 136 that are configured to selectively move the steering wires 134 relative to the catheter end 112. For example, the steering controls 132 may include one or more elements that are configured to move on a track and each element is attached to a corresponding one of the steering wires 134. Moving the elements along the tracks causes the steering wires 134 to move relative to the catheter end 112 which, in turn, causes the catheter to selective bend. For simplicity and brevity, only the driver 136 will be discussed therein. However, it is noted that the mechanisms for moving the driver 136 (as will be discussed in more detail with regards to FIGS. 2A and 2B) will apply to any other element configured to move the steering wires 134 relative to the catheter end 112.

When the driver 136 is disposed in the interior of the handpiece 104, the steering controls 132 may include a mechanism on the exterior of the handpiece 104 that may be manipulated by the user which, in turn, causes the driver 136 to rotate. In an example, as illustrated, the steering controls 132 includes a lever 138 attached to the driver 136. The lever 138 extends along an exterior of the handpiece 104. The lever 138 is configured such that moving the lever 138 relative to the control end 114 causes the driver 136 to rotate thereby selectively bending at least a portion of the catheter 106. In an example, the steering controls 132 includes one or more gears attached to and configured to rotate the driver 136 instead of or in addition to the lever 138. The gears may be attached to a disk-like device that is at least partially exposed on the exterior of the handpiece 104. Rotation of the disk-like device by the user may cause the driver 136 to rotate. In an example, the steering controls 132 includes a motor attached to and configured to rotate the driver 136 instead of or in addition to the lever 138. The motor may be controlled by the host machine 146, the computer 150, the controls 154, or another device.

The catheter 106 of the ureteroscope 102 may be used for imaging an interior surface of a tubular structure, such as a lumen in the body of human or animal. For example, the catheter 106 may be inserted via a subject's urethra to access various parts of the urinary tract. However, it should be appreciated that the ureteroscope 102 may be employed as an industrial endoscope when tubular structure is a part of an industrial apparatus, an equipment, a product, a machine, a production line, and the like. In some embodiments, the catheter 106 may serve as a tether, and may include a plurality of scale markings or fiducials that enable a physician to measure a distance traveled by optoelectronic module 140 into the tubular structure, such as a lumen of a body. Other structure(s) may be built into the ureteroscope 102 as desired.

The ureteroscope 102 may include an optoelectronic module 140 (e.g., a camera or other imager) for imaging the interior of the subject. For example, an optoelectronic module 140 and at least one light source 1142 may be located in a distal end region 110 of the catheter 106 or other location in the catheter 106. The optoelectronic module 140 may include a micro camera module having an image sensor microchip, a set of micro lenses, and a micro illumination module. Suitable optoelectronic modules are disclosed in U.S. Pat. No. 9,942,452, which is incorporated herein, in its entirety, by this reference. In some embodiments, the optoelectronic module 140 may be positioned in a rigid or semi-rigid shell-like housing at the distal end region 110 configured for insertion into the tubular structure for imaging its interior surface. For example, the optoelectronic module 140 may be inserted into a patient's body through a natural body orifice, such as the mouth, nose, urethra, bladder, vagina, or anus. The ureteroscope 102 may therefore have different configurations for use as a gastrointestinal, a colonoscope, endoscopic ultrasound (EUS), endoscopic retrograde cholangiopancreatography, or other suitable application. Applications of the ureteroscope 102 include diagnostic observation associated with endometrial polyps, infertility, abnormal bleeding, and pelvic pain, and surgical procedure such as embryo growth arrest and uterine malformation etc.

The ureteroscope 102 may include a communication interface 144 generally located outside the catheter 106 for receiving the signal from an image sensor within the optoelectronic module 140. The communication interface 144 may be positioned within or adjacent to the handpiece 104. At least the optoelectronic module 140 may be coupled to the communication interface 144.

The endoscopic system 100 may include one or more electronic devices for processing and displaying the image data received from the optoelectronic module 140 of the ureteroscope 102. For example, the endoscopic system 100 may include one or more of a host machine 146 having a microprocessor, a computer 150 having a microprocessor, and a display 148. The host machine 146 may be connected to one or more terminals of the computer 150 and the display 148 for further processing and displaying the image data from the optoelectronic module 140. The host machine 146 or the computer 150 may be programmed with image processing software that takes as input the image data output from the optoelectronic module 140 of the ureteroscope 102 and generates two- or three-dimensional reconstructions of the body lumen that may be displayed on the display 148. Accordingly, a processor in at least one of the host machine 146, the computer 150, or the display 148 may be programmed with software that accepts as input a plurality of still images of an object generated by the optoelectronic module 140, and then output for displaying a three-dimensional rendering of the object based on the plurality of still images. The display 148 may include any suitable display, and may be configured to display a moving image (movie) or a still image collected by the image sensor of the optoelectronic module 140. Although shown in FIG. 1A as separate blocks, the host machine 146, the computer 150, and the display 148 may include a single device, two devices, three devices, or more than three devices.

The endoscopic system 100 also may include a cable 152 configured to operably couple the ureteroscope 102 to at least one of the host machine 146, the computer 150, or the display 148. The cable 152 may electrically couple the communication interface 144 of the ureteroscope 102 to at least one of the host machine 146, the computer 150, or the display 148. The cable 152 also may allow the communication interface 144 to communicate with and receive electric power from the host machine 146 or other power sources. The cable 152 also may be configured to allow the communication interface 144 to transmit image data captured at the optoelectronic module 140 to the host machine 146 for processing, storing, and displaying.

The communication interface 144 may contain, for example, one or more of a processor board, a camera board and frame grabber, or a power source. The processor board may be coupled by the cable 152 to the host machine 146 for storage and retrieval of images generated by ureteroscope 102. The communication interface 144 also may be configured to communicate with and receive electric power from the host machine 146 or other power source via the cable 152. The communication interface 144 also may transmit image data captured at the distal end region 110 to the host machine 146 for processing, storing, and displaying.

The ureteroscope 102 may include one or more controls 154 positioned at or proximate to the control end 114 of the handpiece 104. The one or more controls 154 may include one or more of a switch, a button, a rotatable knob, a movable tab, and the like. In some embodiments, the one or more controls 154 are configured to adjust views presented on the display 148. For example, the one or more controls 154 may be configured to adjust at least one of a brightness, a zoom, a focus or a contrast of one or more images displayed on the display 148. Accordingly, the one or more controls 154 allow a user to adjust views presented on the display 148 and/or computer 150 according to the user's preference and as necessary during use of the ureteroscope 102. In some embodiments, at least one of the one or more controls 154 is configured to activate (e.g., turn on) or deactivate (e.g., turn off) at least one light source 1142 (FIG. 1C) at the distal end region 110 of the catheter 106. In some embodiments, at least one of the one or more controls 154 is configured to activate and deactivate the optoelectronic module 140.

The ureteroscope 102 may be operated to perform or complete selected tasks manually, automatically, or a combination thereof. Some ureteroscopic functions may be implemented with the use of components that comprise hardware, software, firmware or combinations thereof. While general-purpose components such as general purpose computers or oscilloscopes may be used in the ureteroscope 102, dedicated or custom components such as circuits, integrated circuits or software may be too. For example, some functions are implemented with a plurality of software instructions executed by one or more data processors, which is part of a general-purpose or custom computer. The one or more data processors may be in at least one of the communication interface 144, the host machine 146, the computer 150, or the display 148. In some embodiments, the data processor or computer 150 comprises volatile memory for storing instructions and/or data and/or a non-volatile storage, for example, a magnetic hard-disk and/or removable media, for storing instructions and/or data. In some embodiments, implementation includes a network connection. In some embodiments, implementation includes a user interface, generally comprising one or more input devices (e.g., allowing input of commands and/or parameters) and output devices (e.g., allowing reporting parameters of operation and results).

Alternatively, the handpiece 104 may further include a compact battery module for supplying power to the optoelectronic module 140 and the at least one light source 1142. The power source in the handpiece 104 may be, for example, one or more conventional dry-cell disposable batteries or lithium ion rechargeable batteries.

Other examples of endoscopic systems and endoscopes are disclosed in International Publication No. WO2020123588 published on Jun. 18, 2020 and International Application No. PCT/US2022/015828 filed on Feb. 9, 2022, the disclosure of each of which is incorporated herein, in its entirety, by this reference.

As previously discussed, the ureteroscope 102 is configured to cause at least a portion of the catheter 106 to selectively change a rigidity thereof. For example, the ureteroscope 102 is configured to cause the distal end region 110 of the catheter 106 to selectively change a rigidity thereof. In the illustrated embodiment, the ureteroscope 102 is configured to use the working assembly 125 to increase the rigidity of at least a portion of the catheter 106. As will be discussed in more detail below, the working assembly 125 includes a first moveable portion. As used herein, the “first moveable portion” refers to the portions of the working assembly 125 that may move relative to the handpiece 104, such as move relative to the catheter end 112 of the handpiece 104. The first moveable portion may include all of or a portion of the working assembly 125. The first moveable portion of the working assembly 125 is configured to move relative to at least the catheter end 112 of the handpiece 104. Moving the first moveable portion of the working assembly 125 relative to the catheter end 112 may put at least a portion of the working assembly 125 in tension. Putting at least a portion of the working assembly 125 in tension causes the working channel 127 to apply a compressive force to the catheter 106 (e.g., pull the distal end region 110 of the catheter 106 directly back towards the handpiece 104). The compressive force applied to the catheter 106 causes the rigidity of at least a portion of the catheter 106 to increase. For example, the compressive force applied to the catheter 106 causes at least the portion of the catheter 106 at or near the distal end region 110 to exhibit the increased rigidity.

The handpiece 104 include an actuator 156 coupled (e.g., attached to) a portion of the working assembly 125. The actuator 156 is configured to move relative to the rest of the handpiece 104 (e.g., relative to the catheter end 112) responsive to manipulation by a user. The movement of the actuator 156 relative to the catheter end 112 causes at least a portion of the working assembly 125 to also move relative to the handpiece 104. For example, movement of the actuator 156 may cause at least one of at least a portion of the working channel port 126, at least a portion of the working channel 127, at least a portion of the working channel connector 128, or at least a portion of the conduit 130 to move relative to at least a portion of the handpiece 104. The first moveable portion of the working assembly 125 includes the actuator 156 and the other portions of the working assembly 125 that movement of the actuator 156 causes to move.

The actuator 156 may have a first (i.e., initial) position relative to the rest of the handpiece 104. Manipulation (e.g., rotating, pushing, pulling, etc.) of the actuator 156 by the individual may move the actuator 156 to a second position relative to a portion of the handpiece 104 (e.g., the catheter end 112) that is different than the first position. The second position may be further or closer to a portion of the handpiece 104 (e.g., further or closer to the catheter end 112) than the first position. After moving the actuator 156 to the second position, the user may optionally further manipulate the actuator 156 to cause the actuator 156 to return to the first position or move to one or more additional positions that are different than the first and second positions.

In an example, the first moving portion of the working assembly 125 may be in a first (i.e., initial) state when the actuator 156 is in the first position. Substantially no tensile force or a tensile force may be applied to at least a portion of the working assembly 125 when the first moving portion is in the first state. Moving the actuator 156 from the first position to the second position causes the first moving portion to switch to a second state that is different than the first state. When substantially no tensile force is applied to at least a portion of the working assembly 125 when the first moving portion is in the first state, a tensile force may be applied to at least a portion of the working assembly 125 when the first moving portion is in the second state. When a tensile force is applied to at least a portion of the working assembly 125 when the first moving portion is in the first state, a different tensile force (e.g., a greater or smaller) tensile force or substantially no tensile force is applied to at least a portion of the working assembly 125 when the first moving portion is in the second state. Moving the actuator 156 to the one or more additional positions may cause the first moving portion to exhibit one or more different states. It is noted that, as used herein, the first state, the second state, and the one or more additional states may refer to different positions of the first moving portion relative to a portion of the handpiece 104 (e.g., relative to the catheter end 112) and/or the tensile force applied thereto.

In an embodiment, at least a portion of the actuator 156 is easily accessible by a user of the ureteroscope 102. At least a portion of the actuator 156 may be easily accessible, for example, when at least a portion of the actuator 156 is positioned on or extends from an exterior of the handpiece 104. The user may be able to directly manipulate (e.g., rotated, pressed, pulled, etc.) the actuator 156 when at least a portion of the actuator 156 is easily accessible. The manipulation of the actuator 156 by the user allows the actuator 156 move relative to the rest of the handpiece 104. In an example, as illustrated, at least a portion of the actuator 156 may be attached to, positioned in, or otherwise coupled to the working channel port 126. Since the working channel port 126 is positioned on an exterior of the handpiece 104, positioning the actuator 156 to be attached to, positioned in, or otherwise coupled to the working channel port 126 may allow at least a portion of the actuator 156 to be easily accessible.

In an embodiment, the actuator 156 is not easily accessible. The actuator 156 may not be easily accessible, for example, when the actuator 156 is disposed within the handpiece 104 (e.g., at least a portion of the actuator 156 is attached to, disposed in, or otherwise coupled to the working channel 127 or the optional conduit 130). When the actuator 156 is not easily accessible, the actuator 156 may be indirectly manipulated by the user. Indirectly manipulation of the actuator 156 may include using one or more components of the endoscopic system 100 (e.g., the host machine 146, the computer 150, or the controls 154) to control the actuator 156. For example, manipulation of the components of the endoscopic system 100 (e.g., pressing keys on the computer 150, pressing or otherwise moving the controls 154) may indirectly cause the actuator 156 to move relative to the rest of the handpiece 104.

The actuator 156 may include any actuator that is configured to move relative to a portion of the handpiece 104. In an example, as illustrated, the actuator 156 is a threaded element that is attached to a portion of the handpiece 104. In such an example, manipulating the actuator 156 includes rotating an easily accessible portion of the actuator 156 relative to the handpiece 104. Rotating the actuator 156 in a first direction (e.g., counter-clockwise) may cause the actuator 156 to move away from the rest of the handpiece 104 while rotating the actuator 156 in a second direction (e.g., clockwise) may cause the actuator to more towards the rest of the handpiece 104. In an example, not shown, the actuator 156 includes a plunger that may be pulled outwardly or push into a portion of the handpiece 104. In an example, the actuator 156 includes an element attached to a track and moving the actuator 156 relative to a portion of the handpiece 104 includes moving the element along the track. In an example, the actuator 156 includes a motor, a pneumatic press, a hydraulic press, or other actuator.

FIGS. 1D and 1E are enlarged views of the ureteroscope 102 illustrating a method of selectively changing a rigidity of at least a portion of the catheter 106 using the actuator 156, according to an embodiment. Referring to FIG. 1D, the actuator 156 may be initial provided in the first position. In the first position, the actuator 156 may be fully threaded into the working channel port 126 such that the actuator 156 is as close to the catheter end 112 as possible. The actuator 156 is illustrated (in FIG. 1B) as being attached to at least one or more of the working channel connector 128 or the optional conduit 130. Referring to FIG. 1E, the actuator 156 may be rotated (e.g., counterclockwise) to move the actuator 156 to the second position which, in this embodiment, is away from a portion of the handpiece 104 (e.g., away from the catheter end 112). Moving the actuator 156 to the second position moves the first moving portion of the working assembly 125 (e.g., the working channel connector 128, at least a portion of the optional conduit 130, and optionally at least a portion of the working channel 127) from the first state to the second state. In the illustrated embodiment, moving the first moving portion to the second state increases a tensile force that is applied to the working assembly 125. As such, the working channel 127 applies a compressive force to the catheter 106 which increases a rigidity of at least a portion of the catheter 106. Moving the actuator 156 back to the first position may switch the first moving portion of the working assembly 125 back to the first state thereof thereby decreasing the rigidity of at least a portion of the catheter 106.

Other portions of the ureteroscopes disclosed herein other than or in addition to the working assembly 125 may be used to change the rigidity of the catheter. For examples, FIGS. 2A and 2B are side elevational views of a ureteroscope 202 configured to use the steering controls 232 to change the rigidity at least a portion of the catheter 206, according to an embodiment. Except as otherwise disclosed herein, the ureteroscope 202 is the same or substantially similar to any of the ureteroscopes disclosed herein. For example, the ureteroscope 202 may include a handpiece 204, a catheter 206, a working assembly 225 (e.g., at least a working channel port 226 and a working channel), and steering controls 232. The ureteroscope 202 may be used in any of the endoscopic systems disclosed herein.

As previously discussed, the ureteroscope 202 is configured to cause at least a portion of the catheter 206 (e.g., the distal end region of the catheter 206) to selectively change a rigidity of at least a portion thereof. For example, the ureteroscope 202 is configured to use at least a portion of the steering controls 232 to increase the rigidity of at least a portion of the catheter 206. As will be discussed in more detail below, the steering controls 232 includes a second moveable portion. As used herein, the “second moveable portion” refers to the portions of the steering controls 232 that may move relative to the handpiece 204. The second moveable portion may include all of or a portion of the steering controls 232. The first moveable portion of the steering controls 232 is configured to move relative to the catheter end 212 of the handpiece 204. Moving the second moveable portion of the steering controls 232 relative to the catheter end 212 may put at least a portion of the steering wires (shown in FIG. 1B) in tension by moving at least a portion of the steering controls 232. The steering wires are rigidly (i.e., non-moveably) attached to the distal end region of the catheter 206. Thus, putting at least a portion of the steering wires in tension causes the steering wires to apply a compressive force to the catheter 206 (e.g., pull the distal end region of the catheter 206 directly back towards the handpiece 204). The compressive force applied to the catheter 206 causes the rigidity of the catheter 206 to increase.

The handpiece 204 include an actuator 256 that, except as otherwise disclosed herein, is the same as or substantially similar to the actuator 256 of FIGS. 1A-1E. For example, the actuator 256 is coupled (e.g., attached to) a portion of the steering controls 232. The actuator 256 is configured to move relative to the rest of the handpiece 204 (e.g., relative to the catheter end 212) responsive to manipulation by a user. Movement of the actuator 256 causes at least a portion of the steering controls 232 to also move. For example, movement of the actuator 156 may cause at least one of at least a portion of the steering wires, at least a portion of the driver, at least a portion of the lever 238, or any other component of the steering controls 232 to move. The second moveable portion of the steering controls 232 includes the actuator 256 and the other portions of the steering controls 232 that movement of the actuator 256 causes to move.

The actuator 256 may have a first (i.e., initial) position relative to the rest of the handpiece 204, as shown in FIG. 2A. Manipulation of the actuator 256 by the individual may move the actuator 256 to a second position relative to the rest of the handpiece 204 that is different than the first position, as shown in FIG. 2B. The second position may be further or closer to a portion of the handpiece 204 (e.g., further or closer to the catheter end 212) than the first position. After moving the actuator 256 to the second position, the user may optionally further manipulate the actuator 256 to cause the actuator 256 to return to the first position or move to one or more different positions that are different than the first and second positions.

Moving the actuator 256 relative to the catheter end 212 moves at least a portion of the steering controls 232. In other words, moving the actuator 256 causes the second moveable portion of the steering controls 232 to move relative to the handpiece 204. The second moving portion of the steering controls 232 may be in a first (i.e., initial) state when the actuator 256 is in the first position and a second state that is different than the first state when the actuator 256 is in the second position. The second moving portion may also exhibiting one or more additional states when the actuator 256 is in one or more additional positions, as previously discussed.

In an embodiment, at least a portion of the actuator 256 is easily accessible by a user of the ureteroscope 202. At least a portion of the actuator 256 may be easily accessible, for example, when at least a portion of the actuator 256 is positioned on or extends from an exterior of the handpiece 204. In an example, as illustrated, the actuator 256 may include a nob 258 positioned on an exterior of the handpiece 204 thereby allowing the nob 258 to be easily accessible. In an embodiment, the actuator 256 is not easily accessible. The actuator 256 may not be easily accessible, for example, when the actuator 256 is disposed within the handpiece 204 (e.g., at least a portion of the actuator 256 is attached to, disposed in, or otherwise coupled to the driver). When the actuator 256 is not easily accessible, the actuator 256 may be indirectly manipulated by the user.

As previously discussed, in an embodiment, the actuator 256 includes a nob 258. The nob 258 may be directly attached to the second moving portion or may include at least one additional element extending from the nob 258 to the steering controls 232. The portions of the actuator 256 attached to the steering controls 232 may be rotatably attached to the steering controls 232 thereby allowing the driver (not shown) to rotate. The handpiece 204 may define a slot 260 that allows the nob 258 to move side-to-side. Moving the nob 258 side-to-side along the slot 260 allows the actuator 256 to move at least a portion of the steering controls 232. In an embodiment, the actuator 256 may include any of the other actuators disclosed herein, such as a threaded attachment, a plunger that may be pulled outwardly or push into a portion of the handpiece 204, a motor, a pneumatic press, a hydraulic press, or other actuator.

Referring to FIG. 2A, the actuator 256 may be initial provided in the first position. Referring to FIG. 2B, the actuator 256 may be moved to the second position which, in this embodiment, is further away from the catheter end 212. Moving the actuator 256 to the second position moves the second moving portion of the steering controls 232 from the first state to the second state. Moving the second moving portion to the second state increases a tensile force that is applied to the steering wires. The steering wires apply to compressive force to the catheter 206 which increases a rigidity of at least a portion of the catheter 206. Moving the actuator 256 back to the first position may switch the second moving portion of the steering controls 232 back to the first state thereof thereby decreasing the rigidity of at least a portion of the catheter 206.

It is noted that other components of the ureteroscopes disclosed herein may be used to change the rigidity of at least a portion of the catheter. In an example, the ureteroscopes disclosed herein may include one or more actuators configured to apply a tensile force directly to the steering wires with or without moving the steering controls. In an example, the ureteroscopes disclosed herein may include one or more actuators configured to apply a tensile force to wires extending from the optoelectronic module 140 and/or the light sources 142 of FIG. 1C to apply a compressive force to the catheter. In an example, the ureteroscopes disclosed herein may include a laser fiber and/or a laser fiber channel configured to receive the laser fiber, as disclosed in International Application No. PCT/US2022/015828 filed on Feb. 9, 2022, the disclosure of which was previously incorporated herein. In such an example, one or more actuators may be configured to apply a tensile force to the laser fiber and/or the laser fiber channel thereby applying a compressive force to the catheter.

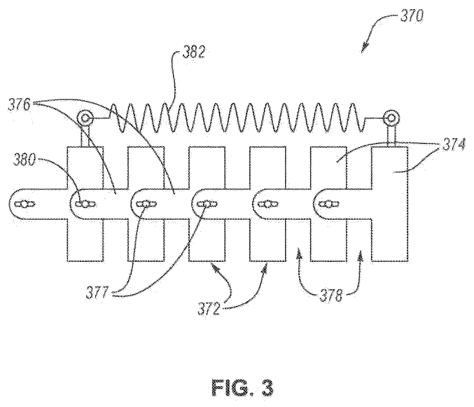

Catheter shafts are often included in at least the distal end regions of catheters. Catheter shafts are included in catheters to provide support to such regions of the catheters and to better control bending of the catheters. In an embodiment, the catheters disclosed herein may include a catheter shaft that is configured to facilitate changing the rigidity thereof. For example, FIG. 3 is a side elevational view of a catheter shaft 370 that may be used in any of the catheters disclosed herein, according to an embodiment. The catheter shaft 370 includes a plurality of links 372. The plurality of links 372 may be substantially hollow (e.g., generally annular) such that a working channel, steering wires, and other components of the catheter may be positioned therethrough. The links 372 may be pivotably attached to adjacent ones of the links 372 thereby allowing the catheter shaft 370 to pivot as the catheter bends. For example, each of the links 372 may include a body 374. At least some of the links 372 also include an attachment portion 376 extending from the body 374. The attachment portion 376 may be attached to an adjacent body 374 in a manner that allows the adjacent bodies 374 to move (e.g., pivot) relative to each other. In an example, as illustrated, the attachment portion 376 may be attached to an adjacent body 374 using a hinge 377. In an example, the attachment portion 376 is integrally formed with or rigidly attached to the body 374 of an adjacent link 372. In such an example, the attachment portion 376 is elastic thereby allowing the adjacent bodies 374 to pivot relative to each other by deforming the attachment portion 376.

At least a portion of each of the adjacent links 372 are separated from each other by a gap 378. For example, the gap 378 may be located between adjacent bodies 374. The catheter shaft 370 may be configured such that the size of the gap 378 between the adjacent links 372 (i.e., the distance between adjacent links 372) may change. In an example, as illustrated, the attachment portions 376 define elongated slots 380 that are configured to change the size of the gap 378. In particular, the hinge 377 may be disposed in the slot 380 and may move back and forth in the slot 380 thereby increasing or decreasing the size of the gaps 378. In an example, the attachment portions 376 may be configured to deform to change the size of the gap 378.

Changing the size of the gaps 378 may make the catheter shaft 370 more or less rigid. For example, the movement between the links 372 may be limited by adjacent bodies 374 contacting each other. Increasing the size of the gaps 378 increases the amount of movement between adjacent bodies 374 before adjacent bodies 374 contact each other. In other words, increasing the size of the gaps 378 decreases the rigidity (i.e., increases the flexibility) of the catheter shaft 370 due to the increased allowable movement between the adjacent bodies 374. However, decreasing the size of the gaps 378 decreases the amount of movement between adjacent bodies 374 before adjacent bodies 374 contact each other. In other words, decreasing the size of the gaps 378 increases the rigidity (i.e., decreases the flexibility) of the catheter shaft 370 due to the increased allowable movement between the adjacent bodies 374.

The size of the gap 378 may depend on whether a compressive force is applied to the catheter. For example, applying a compressive force to the catheter decreases the size of the gaps 378 thereby increasing the rigidity of the catheter shaft 370. Increasing the rigidity of the catheter shaft 370 increases the rigidity of the portions of the catheter than includes the catheter shaft 370. Decreasing or removing the compressive force applied to the catheter allows the size of the gaps 378 to increase thereby decreasing the rigidity of the catheter shat 370 and the portions of the catheter including the catheter shaft 370.

The catheter shaft 370 may be configured to increase the size of the gap 378 when the compressive force applied to the catheter decreases or is removed. In an example, the catheter shaft 370 may include a spring 382 (schematically illustrated in FIG. 3) connected to one or more of the links 372 (e.g., connected to the links 372 at the terminal ends of the catheter shaft 370). The spring 382 may apply a biasing force that pushes the links 372 apart when a compressive force applied to the catheter is decreased or is removed. In an example, the catheter shaft 370 does not include a spring. In such an example, the resiliency (i.e., the desire to return to the original shape) of the catheter shaft 370 and/or the catheter than includes the catheter shaft 370 increases the size of the gap 378 without needing a spring.

It is noted that the ureteroscopes disclosed herein may be used in endoscopic procedures that do not involve the urinary tract (e.g., at least one of the urethra, the bladder, the ureter, or the kidney). For example, the ureteroscopes may be used in endoscopic procedures that involve the gastrointestinal tract, the respiratory tract, the ear, the reproductive system, the abdominal or pelvic cavity, the interior of a joint, the organs of the chest, a fetus, the hand, or any other location instead of or in addition to the urinary tract.

While various aspects and embodiments have been disclosed herein, other aspects and embodiments are contemplated. The various aspects and embodiments disclosed herein are for purposes of illustration and are not intended to be limiting.

Terms of degree (e.g., “about,” “substantially,” “generally,” etc.) indicate structurally or functionally insignificant variations. In an example, when the term of degree is included with a term indicating quantity, the term of degree is interpreted to mean ±10%, ±5%, or +2% of the term indicating quantity. In an example, when the term of degree is used to modify a shape, the term of degree indicates that the shape being modified by the term of degree has the appearance of the disclosed shape. For instance, the term of degree may be used to indicate that the shape may have rounded corners instead of sharp corners, curved edges instead of straight edges, one or more protrusions extending therefrom, is oblong, is the same as the disclosed shape, etc.

1. A endoscope, comprising:
a handpiece including:
a catheter end;
a control end;
a working assembly configured to receive one or more instruments;
steering controls configured to bend at least a portion of the catheter;
a moveable portion configured to move relative to the catheter end of the handpiece; and
a catheter extending from the catheter end of the handpiece, the catheter including a proximal end region and a distal end region spaced further from the handpiece than the proximal end region;
wherein movement of the moveable portion relative to the catheter end of the handpiece adjusts a rigidity of a portion of the catheter by compressing the catheter or compressing a catheter shaft attached to the distal end of the catheter; and
wherein at least one of the working assembly or the steering controls includes the moveable portion.

2. The endoscope of claim 1, wherein a rigidity of the distal end region of the catheter increases when the at least one of at least a portion of the first moveable portion moves further from the catheter end.

3. The endoscope of claim 1, wherein the working assembly includes the moveable portion.

4. (canceled)

5. The endoscope of claim 3, wherein the moveable portion includes a threaded actuator threadedly attached to the handpiece, and wherein rotating the threaded actuator increases or decreases a distance measured from the actuator to the catheter end of the handpiece.

6. The endoscope of claim 1, wherein the steering controls includes the moveable portion.

7. (canceled)

8. The endoscope of claim 6, wherein the moveable portion includes a nob extending from at least a portion of the steering controls.

9. The endoscope of claim 1, wherein the catheter includes the catheter shaft, the catheter shaft including a plurality of links, each of the plurality of links pivotally attached to adjacent ones of the plurality of links, at least a portion of the adjacent ones of the plurality of links separated by a gap, movement of the moveable portion relative to the catheter end of the handpiece increases a rigidity of a portion of the catheter by compressing the catheter shaft.

10. The endoscope of claim 9, wherein the plurality of links are configured to have the gap between adjacent ones of the plurality of links decreased when at least a portion of the moveable portion moves away from the catheter end.

11. The endoscope of claim 9, wherein the catheter shaft includes at least one spring attached to at least some of the plurality of links.

12. A method of using a endoscope, the method comprising:
providing the endoscope, the endoscope including:
a handpiece including:
a catheter end;
a control end;
a working assembly configured to receive one or more instruments;
steering controls configured to bend at least a portion of the catheter; and
a catheter extending from the catheter end of the handpiece, the catheter including a proximal end region and a distal end region spaced further from the handpiece than the proximal end region; and
moving at least a portion of a moveable portion relative to the catheter end of the handpiece of the endoscope to adjust a rigidity of at least a portion of the catheter by compressing the catheter or compressing a catheter shaft attached to the distal end of the catheter;
wherein at least one of the working assembly or the steering controls includes the moveable portion.

13. The method of claim 12, wherein moving the moveable portion relative to the catheter end of the handpiece includes moving the first moveable portion away from the catheter end to increase the rigidity of the distal end of the catheter.

14. The method of claim 12, wherein moving the moveable portion relative to the catheter end of the handpiece includes moving at least a portion of the moveable portion towards the catheter end to decrease the rigidity of the distal end of the catheter.

15. The method of claim 12, wherein the working assembly includes the moveable portion.

16. The method of claim 15, wherein moving at least a portion of the moveable portion relative to the catheter end includes rotating a threaded actuator that is attached to the handpiece.

17. The method of claim 12, wherein the steering controls includes the moveable portion.

18. The method of claim 17, wherein moving at least a portion of the moveable portion relative to the catheter end includes moving a nob coupled to the steering controls relative to the catheter end.

19. The method of claim 12, wherein moving at least a portion of the moveable portion includes decreasing a gap between adjacent ones of a plurality of links of the catheter shaft disposed within the catheter, each of the plurality of links pivotally attached to the adjacent ones of the plurality of links.

20. The method of claim 12, wherein moving at least a portion of the moveable portion includes increasing a gap between adjacent ones of a plurality of links of the catheter shaft disposed within the catheter, each of the plurality of links pivotally attached to the adjacent ones of the plurality of links.

21. The endoscope of claim 1, wherein the working assembly includes a working channel port and, optionally, a working channel connector connected to the working channel port or a conduit at least attaching the working channel port or the working channel connector to a working channel of the catheter;
wherein the moveable portion is connected to at least one of the working channel port, the working channel connector, or the conduit; and
wherein movement of the moveable portion relative to the catheter end of the handpiece moves at least one of at least one of the working channel port, the working channel connector, or the conduit to change a rigidity of the portion of the catheter b y compressing the catheter or compressing the catheter shaft.

22. The endoscope of claim 3, wherein the moveable portion defines a channel configured to receive the one or more instruments.
